(19) United States
(12) Patent Application Publication
(10) Pub. No.: US 2019/0110981 A1
(43) Pub. Date: Apr. 18, 2019

(54) Cannabinoid Patch

(71) Applicant: LIFE TECH GLOBAL, LLC, Irvine, CA (US)

(72) Inventor: Ludwig Weimann, San Diego, CA (US)

(21) Appl. No.: 16/163,410

(22) Filed: Oct. 17, 2018

Related U.S. Application Data
(60) Provisional application No. 62/573,609, filed on Oct. 17, 2017.

(51) Int. Cl. A61K 9/00; A61K 9/70; A61K 31/05; A61K 31/352

(52) U.S. Cl. CPC A61K 9/006; A61K 9/0014; A61K 31/4045; A61K 31/05; A61K 31/352; A61K 9/7084

(57) ABSTRACT

The disclosure provides pharmaceuticals, cannabinoids, and nutraceuticals and dermal patches, as well as chemicals that enhance delivery of pharmaceuticals from a buccal patch or from a dermal patch, where the patch may include a film, adhesive, emulsifier, tackifier, or hydrogel.

### CROSS REFERENCE TO RELATED APPLICATIONS

This application claims the benefit of and priority to U.S. Provisional Patent Application Ser. No. 62/573,609, filed Oct. 17, 2017, the content of which is incorporated herein by reference herein in its entirety.

### FIELD OF THE DISCLOSURE

The disclosure relates to sublingual patches, tablets, capsules, and pills, as well as to buccal patches and dermal patches, each of which can contain a formulation providing a pharmaceutical agent such as a drug or a nutraceutical. The drug can be one or more cannabinoids.

### BACKGROUND OF THE DISCLOSURE

Dermal patches can take the form of a monolithic-style patch or a reservoir-style patch (see, US 2017/0071870 of Weimann, which is hereby incorporated herein in its entirety). Monolithic-style patch can take the form of a sandwich, where the face that is exposed to the atmosphere is a backing, where the opposite face is a release liner, and where the filling of the sandwich is a matrix that includes an adhesive and a pharmaceutical agent such as a drug or nutraceutical. Prior to applying the patch to the skin, a release liner is removed and discarded.

Regarding reservoir-style patch, the reservoir can contain a pharmaceutical agent that is a drug or a nutraceutical. The reservoir also contains a liquid carrier and a gelling agent. The reservoir can be defined by a backing and by a permeable membrane, which together assume a “ravioli” conformation. The permeable membrane is optionally coated with an adhesive that mediates binding of the adhesive to the skin. On one side of the adhesive is the permeable membrane, and on the other side is a release linter. Prior to applying the patch to the skin, a release liner is removed and discarded.

Dermal patches are used to deliver capsaicin for reducing pain. The patch delivers capsaicin. Capsaicin acts on peripheral nociceptors. The patch can be applied for about one hour, where the result is pain reduction for many weeks (see, Peppin et al (2011) J. Pain Res. 4:385-392). Dermal patches are also used to deliver torigotine for treating Parkinson's disease, and where the patch provides continuous drug delivery over 24 hours, resulting in plasma pharmacokinetics similar to that with continuous i.v. infusions. Rotigotine acts on dopamine receptors (see, Elshoff et al (2015) Drugs. 75:487-501). To give another example, dermal patches can provide estrogen for therapy to post-menopausal women, and to provide ethinyl estradiol and norelgestromin for contraception. The contraceptive patch is used for 7 days, and it provides systemic concentrations similar to that with a daily oral contraceptive (see, Jung et al (2013) Drugs. 13:223-233).

The present disclosure provides sublingual tablets, capsules, pills, and strips, as well as buccal patches and dermal patches. These objects are provided herein as novel and enhanced tablets, capsules, strips, and patches that contain one or more drugs. Also provided are these same novel and enhanced objects, that do not contain one or more drugs, for example, as might find use as a placebo.

The present disclosure addresses an unmet need for sublingual tablets, capsules, and pills, dermal patches, sublingual patches, and buccal patches that provide pharmaceutical agents such as a cannabinoid, melatonin, capsaicin, lidocaine, salicylic acid, sildenafil, or a vitamin such as vitamin B1, vitamin D3, vitamin B12, or vitamin C.

### SUMMARY OF THE DISCLOSURE

Briefly stated, the present disclosure provides a composition capable of use in a buccal patches, sublingual patch, pill, tablet, or a dermal patch, wherein the composition comprises one or more of, an acrylic adhesive with non-functionality and an adhesive with only OH-functionality, further comprising one of more of enhancers selected from azone, oleic acid, and dimethylsulfoxide (DMSO); a polyisobutylene (PIB adhesive) with tackifiers that improve adhesion to skin using acrylic pressure sensitive adhesive mixed in at 1-50%, optionally with a cycloaliphatic hydrocarbon. resin; a PIB adhesive with enhancers: at 3% of azone or oleic acid double the transdermal delivery from PIB; hemp oil with CBD of concentration 80-95% containing at least one terpene; a semisolid hydrogel that is saturated with cannabidiol (CBD) and tetrahydroxannabinol (THC); a semisolid hydrogel comprising an oil that consists essentially of CBD and THC (80-95%, wt/vol), in combination with ethanol/water (80/20, vol/vol), optionally with one or more enhancers selected from azone, oleic acid, and limonene; a semisolid hydrogel saturated with CBD and THC oils (80-95%, wt/vol), wherein the oil is mixed with EtOH/water (80/20, vol/vol), optionally with one or more enhancers selected from azone, oleic acid, and limonene; or a THC oil of THC (80-95%) mixed with 1-20% EtOH or with 1-10% EtOH/water (80/20, vol/vol) wherein including greater than 10% of ethanol is capable of lowering flux of THC delivery as determinable with a reservoir patch. Also provided is a buccal patch, sublingual pill, sublingual tablet, or sublingual patch, comprising one of the above compositions.

Moreover, what is further provided is a dermal patch comprising one of the above compositions. In another aspect, what is provided is a method for applying the above buccal patch to the buccal mucosa of a human subject, and allowing a cannabinoid to transit from the buccal patch into the buccal mucosa of the human subject. Also provided, is a method for applying the above dermal patch to skin of a human subject, and allowing a cannabinoid to transit from the buccal patch into the skin of the human subject.

What is also embraced is a method for manufacturing the above patch, comprising the steps of combining THC, a film, an adhesive, and a backing, to generate an uncut patch, further comprising the uncut patch to produce a cut patch that is capable of applying to human skin or of applying to human buccal pouch.

In embodiments, the present disclosure provides a method for delivering cannabidiol (CBD) to human skin, wherein the method comprises the step of contacting a dermal patch with the human skin, and wherein the contacting is for at least one hour, and wherein the delivering results in passage of CBD through the skin, wherein the method uses a dermal patch that comprises polyisobutylene (PIB) and a cannabis oil that contains about 15% cannabidiol (CBD) oil, and wherein the delivering is measurable by a system that comprises human cadaver skin and a Franz diffusion cell, wherein said method is capable of delivering one or both of: (i) A cumulative flux of CBD of at least about 20 micrograms CBD per cm² of skin over a period of ten hours, and (ii) A cumulative flux of CBD of at least about 40 micrograms CBD per cm² of skin over a period of twenty hours. In another aspect, what is provided is a method for delivering tetrahydrocannabinol (THC) to human skin, wherein the method comprises the step of contacting a dermal reservoir patch with the human skin, and wherein the contacting is for at least one hour, and wherein the delivering results in passage of THC through the skin, the method resulting in cumulative flux of THC from a saturated ethanol/water solution with 25% THC oil, and wherein one or both of: (i) The ratio of ethanol/water is about 70/30 and the flux is at least about 40 micrograms THC per cm², and (ii) The ratio of ethanol/water is about 60/40 and flux is at least about 50 micrograms THC per cm². Moreover, the present disclosure also provides a sublingual tablet of about 250 mg, wherein the sublingual tablet comprises CBD (20 mg) and melatonin (3 mg), and wherein the sublingual tablet has a friability of about 0.3%, a hardness of about 3 kg/cm2, and a disintegration of about 45 seconds. Also provided is the above sublingual tablet, with an alternative weight, but with the ratio of CBD to melatonin being the same as above (as disclosed above, this ratio is 20 mg CBD/3 mg melatonin). The present disclosure provides a sublingual tablet of about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, or a sublingual tablet that is 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, and the like, or a sublingual tablet with a weight in the range of 25-50 mg, 50-75 mg, 75-100 mg, 100-125 mg, 125-150 mg, 150-175 mg, 175-200 mg, 200-225 mg, 225-250 mg, 250-275 mg, 275-300 mg, 300-325 mg, 325-350 mg, 350-375 mg, 375-400 mg, and so on. In exclusionary embodiments, the present disclosure can exclude any tablet that meets one of the above values, or that meets one of the above ranges, or that is greater than any of the above values or is less than any of the above values. Also provided is one or more of the above tablets (the above tablets in the above weights or in the above weight ranges), where the ratio of CBD/melatonin is 4/3, 8/3, 12/3, 16/3, 20/3, 24/3, 28/3, 32/3, 36/3, and so on, or where the ratio of CBD/melatonin occurs in a range, and where the range is between any of the values of 4/3, 8/3, 12/3, 16/3, 20/3, 24/3, 28/3, 32/3, 36/3. In exclusionary embodiments, the present disclosure can exclude any tablet that meets one of the above values, or that meets one of the above ranges, or that is greater than any of the above values or is less than any of the above values.

### BRIEF DESCRIPTION OF DRAWINGS

FIG. 1. In vitro cumulative flux (micrograms/cm²) of cannabidiol (CBD) through skin versus time (hours). Upper curve is from patch containing CBD oil. Lower curve is from patch containing crystalline CBD.

FIG. 2. In vitro flux of tetrahydrocannabinol (THC) from ethanol/water, at each of the indicated ratios of ethanol/water.

FIG. 3. In vitro cumulative flux (micrograms/cm²) of CBD (upper curve) or THC (lower curve) over the course of about 20 hours, from a patch containing both CBD and THC.

### DETAILED DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual patent, and published patent application, as well as figures, drawings, sequence listings, compact discs, and the like, was specifically and individually indicated to be incorporated by reference. The term “about” can refer, for example, to a range that encompasses a given value plus or minus 2%, a given value plus or minus 4%, a given value plus or minus 8%, a given value plus or minus 12%, and the like. PCT/US14/22054 is incorporated expressly by reference as if fully set forth herein.

Cannabinoids

The present disclosure provides dermal patches, formulations, dermal patches not containing a formulation, and dermal patches including a formulation. Preferred formulations include one or more cannabinoids. The major cannabinoids from cannabis sativa are cannabidiol (CBD), cannabichromene (CBC), cannabigerol (CBG), delta-9-tetrahydrocannabinol (delta-9-THC), and cannabinol (CBN) (Appendino et al (2008) J. Nat. Prod. 71:1427-1430). Clinical trials have established that formulations derived from cannabis, can improve neuropathic pain of multiple sclerosis, improve appetite and sleep quality in cancer patients, relieve pain in fibromyalgia patients, and serve as an anti-emetic for chemotherapy induced nausea and vomiting (see, Health Canada (February 2013) Information for Health Care Professionals. Cannabis (Marihuana, Marijuana) and the Cannabinoids (152 pages)). The present disclosure also provides tetrahydrocannabinovarin (THCV), which is a propyl analogue of THC, and cannabidivarin (CBDV), which is a propyl analogue of CBD.

Formulations and compositions that include both THC and CBD at a given ratio are provided, such as at the ratio of about 95/5, about 90/10, about 80/20, about 70/30, about 60/40, about 50/50, about 40/60, about 30/70, about 20/80, about 10/90, and about 5/95 (by weight). Administering formulations containing both THC and CBD can have greater influence on reducing pain that formulations containing only THC or only placebo (see, Johnson et al (2010) J. Pain Symptom Management. 39:167-179; Notcutt et al (2004) Anaesthesia. 5944-452).

One of more of the following cannabinoids can be included in the compositions of the present disclosure. Cannaboids and related compounds further include, for example, cannabichromene; cannabitriol; cannabicyclolol; cannabielsoin, cannabinodiol; delta-8-tetrahydrocannabinol; cannabichromanone; cannabicoumaronone; cannabicitran; 10-oxo-delta-6a10a-tetrahydrocannabinol; cannabiglendol; delta-7-isotetrahydrocannabinol; CBLVA; CBV; CBEVA-B; CBCVA; delta-9-THCVA; CBDVA; CBGVA; divarinolic acid; quercetin; kaemferol; dihydrokaempferol; dihydroquercetin; cannflavin B; isovitexin; apigenin; naringenin; eriodictyol; luteolin; orientin; cytisoside; vitexin; canniprene; 3,4′-dihydroxy-5-methoxy bibenzyl; dihydroresveratrol; 3,4′-dihydroxy-5,3′-dimethoxy-5′-isoprenyl; cannabistilbene 1; cannabistilbene 11a; cannabistilbene 11b; cannithrene 1; cannithrene 2; cannabispirone; iso-cannabispirone; cannabispirenon-A; cannabispirenone-B; cannabispiradienone; alpha-cannabispiranol; beta-cannabispiranol; acetyl-cannabispirol; 7-hydroxy-5-methoxyindan-1-spiro-cyclohexane; 5-hydroxy-7-methoxyindan-1-spiro cyclohexane; myristic acid, palmitic acid, oleic acid, stearic acid, linoleic acid, linolenic acid, arachidic acid, eicosenoic acid, behenic acid, lignoceric acid, 5,7-dihydroxyindan-1-cyclohexane; cannabispiradienone; 3,4′-dihydroxy-5-methoxybibenzyl; canniprene; cannabispirone; cannithrene I; cannithrene 2; alpha-cannabispiranol; acetyl-cannabispirol; vomifoliol; dihydrovomifoliol; beta-ionone; dihydroactinidiolide; palustrine; palustridine; plus-cannabisativine; anhydrocannabisativine; dihydroperiphylline; cannabisin-A; cannabisin-B; cannabisin-C; cannabisin-D; grossamide; cannabisin-E; cannabisin-F; cannabisin-G; and so on (see, e.g., Flores-Sanchez and Verpoorte (2008) Secondary metabolism in cannabis. Phytochem. Rev. 7:615-639).

In exclusionary embodiments, the present disclosure can exclude any formulation, oil, fluid, composition, device, or method that comprises CBD, CBC), CBG, delta-9-THC, CBN, or any chemical in the above list. The present disclosure can also exclude any formulation, oil, fluid, composition, device, or method that comprises oleic acid, that comprises ethanol, or that comprises both oleic acid and ethanol. In range embodiments, what can be excluded is any one of the above chemicals, where the chemical is dissolved or suspended or dispersed in a liquid, in an oil, in a paste, in an adhesive, and where the chemical occurs at concentration of over 0.05% of the liquid, oil, paste, or adhesive. Also, what can be excluded is any one of the above chemicals, where the chemical is dissolved or suspended or dispersed in a liquid, in an oil, in a paste, in an adhesive, and where the chemical occurs at concentration of over 0.1%, over 0.5%, over 1.0%, over 2%, over 5%, over 10%, over 15%, over 20%, over 25%, over 30%, over 40%, over 50%, over 60%, over 70%, over 80%, or over 90% of the liquid, oil, paste, or adhesive.

Measuring Cannabinoids

Cannabinoids can be separated, purified, analyzed, and quantified by a number of techniques. Available equipment and methods include, e.g., gas chromatography, HPLC (high pressure liquid chromatography, high performance liquid chromatography), mass spectrometry, time-of-flight mass spectrometry, gas chromatography-mass spectrometry (GC-MS), and liquid chromatography-mass spectrometry (LC-MS). Equipment for separation and analysis is available from Waters Corp., Milford, Mass.; Agilent, Foster City, Calif.; Applied Biosystems, Foster City, Calif.; and Bio-Rad Corp., Hercules, Calif.

The present disclosure provides in-line monitoring of purification, that is, quantitation of THC as well as quantitation of impurities. In-line monitoring may be by UPLC methods, or by other methods. Ultra-high performance liquid chromatography (UPLC) is similar to HPLC, except that UPLC uses smaller particles in the column bed, and greater pressures. The particles can be under 2 micrometers in diameter, and pressures can be nearly 15,000 psi. UPLC also uses higher flow rates, and can provide superior resolution and run times in the range of under 30 seconds (Wren and Tchelitcheff (2006) J. Chromatography A. 1119:140-146; Swartz, M. E. (May 2005) Separation Science Redefined). The application of UPLC to cannabinoids has been described (see, Jamey et al (2008) J. Analytical Toxicology. 32:349-354; Badawi et al (2009) Clinical Chemistry. 55:2004-2018). Suitable UPLC columns for cannabinoid analysis include, e.g., Acquity@UPLC HSS T3 C18, and Acquity® UPLC BEH C18 column (Waters, Milford, Mass.). Other methods for detecting cannabinoids include, e.g., infrared (IR) spectroscopy, gas chromatography mass spectroscopy (GCMS), and electrospray tandem mass spectroscopy (ESI-MS/MS) (Ernst et al (2012) Forensic Sci. Int. 222:216-222).

Cannabis oil is available, for example, from Poland. Polish cannabis oil is available from HemPoland, located at ul. S. Sulimy 1, 82-300 Elblag, Poland. Polish cannabis oil is also available from CannabiGold, located at ul. S. Sulimy 1, 82-300 Elblag, Poland.

Biochemical properties of cannabinoids, binding to cannabinoid receptors, terpenes and terpene receptor binding, can be assessed using labeled cannabinoids, labeled terpenes, and labeled ligands where a cannabinoid or a terpene influences binding properties of the labeled ligand. Useful labels include radioactive labels, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

Cannabinoid Numbering Systems

The present disclosure uses the nomenclature as set forth by Pertwee R G et al (2010) International Union of Basic and Clinical Pharmacology. LXXIX. Cannabinoid receptors and their ligands: beyond CB1 and CB2. Pharmacol. Rev. 62:588-631. Regarding different numbering systems for the same compound, AVIV (US 2004/0110827) states that: “It should be noted that for historical reasons, these cannabinoid analogs are still named following the previous nomenclature, where the terpenic ring was the base for the numbering system. Then the chiral centers of THC type cannabinoids were at carbon atoms 3 and 4. The accepted nomenclature is now based on the phenolic ring as the starting point for numbering. Thus, THC that was previously described as delta-1-THC was later renamed delta-9-THC, similarly delta-6-THC was renamed delta-8-THC, and the chiral centers are at carbons 6a and 10a.” AVIV also has this comment about enantiomers: “delta-9-THC was established by Mechoulam R. et al. in 1967 and found to be of (−)-(3R,4R) stereochemistry. It was later found that the psychotropic activity of cannabinoids resides in the natural (3R,4R) OH series, while the opposite enantiomeric synthetic series (3S,4S) was free of these undesirable effects.”

According to Agurell et al (1988) Pharmacological Reviews. 38:21-43, the terpene numbering system uses delta-1-THC, while the dibenzopyran system uses delta-9-THC to refer to the same chemical. Both of these numbering systems can be used for THC, CBD, and CBN.

According to Chulgin, the numbering system most broadly used recognizes both the terpene nature and the aromatic nature of the two different parts of the cannabinoid. Here, the terpene is numbered from the ringcarbon that carries that branched methyl group, and this is numbered 7, and the remaining three carbons of the isopropyl group are then numbered sequentially. The advantage to this numbering system is that this numbering system is applicable whether the center ring is closed or open. Other numbering systems are the biphenyl numbering system, the Chemical Abstracts system (substituted dibenzopyran numbering), and the Todd numbering system (pyran numbering) (see, Chulgin A T (1969) Recent developments in cannabis chemistry. J. Psychedelic Drugs. pp. 397-415.

Matrix Embodiments

An excipient useful for granulating agents and sprays is the polyvinylpyrrolidone copolymer having a given ratio, or range of ratios, of polyvinylpyrrolidone/vinyl acetate (PVP/VA). The present disclosure provides PVP/VA (or combinations of any two polymers), at a ratio of 10/90, 20/80, 30/70, 40/60, 50/50, 60/40, 70/30, 80/20, 90/10, as well as a combination of any two polymer at a ratio of about 10/90, about 20/80, about 30/70, about 40/60, about 50/50, about 60/40, about 70/30, about 80/20, about 90/10. Also, the present disclosure can exclude PVP/VA compositions (or it can exclude a combination of any two polymers) with a ratio of, 10/90, 20/80, 30/70, 40/60, 50/50, 60/40, 70/30, 80/20, 90/10, or about 10/90, about 20/80, about 30/70, about 40/60, about 50/50, about 60/40, about 70/30, about 80/20, about 90/10, and the like. The PVP/VA copolymer has the ability to distribute homogeneously around an active ingredient during formation of an aqueous liquid phase (see, US2016/0058866 of Sekura). Polymers and copolymers are available from Sigma-Aldrich, St. Louis, Mo., Nippon Shokubai Co., Ltd., Osaka, Japan, BASF Corp., Florham Park, N.J., and Ashland, Schaffhausen, Switzerland.

In methods of manufacturing embodiments, monolith patch can be made as follows. Cannabis oil or one or more pure cannabinoids can be combined with permeation enhancers such as oleic acid and dodecylmethyl sulfoxide. Then one or more pure terpenes, or an essential oil, or a combination of an essential oil and one or more pure terpenes, is mixed with the above combination. Then, a pressure sensitive adhesive such as silicone adhesive BIO PSA 7-4302 (Dow Corning), or other suitable ones are mixed in. Finally, the mixture is spread into one or more sheets, cured at room temperature for several hours or longer. After drying, a foam backing layer is applied, and then the product is cut into shapes (e.g., squares, rectangles, ovals, round-edged squares or round-edged rectangles, circles) suitable for applying to the skin of a person.

A laminate that can be held in place on the gingiva (gums) takes the form of a semipermeable outer layer, reservoir having a pharmaceutical, backing layer, where the backing layer faces the gingiva. Saliva can enter through the semipermeable outer layer, pass through the reservoir, and then draw medicine into contact with gingiva for absorption in the bloodstream. A pharmaceutical can be freeze dried or can occur as a hydrogel matrix, in the reservoir. The present disclosure provides a backing layer of one or more polymers, such as, ethyl cellulose, butyl cellulose, hydroxybutyl cellulose, or polyvinylalcohol. An amorphous or semi-crystalline excipient matrix can be made from methylcellulose, ethylcellulose, hydroxypropyl methylcellulose, cellulose acetate phthalate, or cellulose acetate butyrate. In exclusionary embodiments, the present disclosure can exclude one or more of these polymers.

In reservoir-distribution embodiments, a pharmaceutical or nutraceutical can be distributed evenly throughout reservoir, or can be distributed at a higher concentration at center of reservoir, or can be distributed at a higher concentration at region of reservoir that is closer to the skin when patch is situated and adhering to skin.

Hydrogels

Hydrogels are 3-dimensional, cross-linked networks of water-soluble polymers. The porous structure of hydrogels can be altered by changing the density of cross-linking. The degree of cross-linking can alter the rate of loading a drug, and it can alter the rate of drug release. The present disclosure can encompass a hydrogel that consists of one of the following polymers or alternatively, that comprises one or more of the following polymers (e.g., as a block polymer). The polymers include, poly(ethylene oxide) (PEO), poly(propylene oxide) (PPO), poly(lactide-co-glycolic acid) (PLGA), poly(N-isopropylacrylamide) (PNIPAM), poly(propylene fumarate) (PPF), poly(caprolactone) (PCL), poly(urethane) (PU), and poly(organophosphazene) (POP). An example of a block polymer is PEO-PPO-PEO. In exclusionary embodiments, the present disclosure can exclude a hydrogel that includes PEO, PPO, PLGA, PNIPAM, PPF, PCL, PU or POP. The present disclosure also encompasses hydrogels that contain a cyclodextrin, where the cyclodextrin is cross-linked to hydrogel (see, Hoare et al (2008) Hydrogels in drug delivery: Progress and challenges. Polymer. 49:1993-2007). Hydrogels of the present disclosure can be ethylene vinylacetate, alginic acid, gums, polyvinylalcohol hydrogel; silicone hydrogel; polyvinylalcohol/dextran hydrogel; alginate hydrogel; alginate-pyrrole hydrogel; gelatin/chitosan hydrogel; polyacrylic acid hydrogel; photo crosslinked polyacrylic acid hydrogel; amidated pectin hydrogel; pectin hydrogel; gelatin hydrogel; polyethylene glycol (PEG) hydrogel; carboxymethylcellulose/gelatin hydrogel; chitosan hydrogel, as well as mixtures thereof, or copolymers thereof, and the like.

Cyclodextrins

Cyclodextrins are cyclic oligosaccharides of (alpha-1,4)-linked alpha-D-glucopyranose units, with a lipophilic central cavity and a hydrophilic outer surface. As a result of their molecular structure and shape, they can act as molecular containers by trapping drugs or other molecules in their internal cavity. No covalent bonds are formed or broken during drug cyclodextrin complex formation, and in aqueous solution, the complexes readily dissociate and free drug molecules remain in equilibrium with the molecules bound within the cyclodextrin cavity (see, Tiwari et al (2010) Cyclodextrins in delivery systems: Applications. J. Pharm. Bioallied Sci. 2:72-79). Derivatives of cyclodextrins that are hydroxypropyl (HP), methyl (M) and sulfobutylether (SBE) substituents are useful as pharmaceutical excipients.

Cyclodextrins for use, for example, in cannabinoid/cyclodextrin complex, include beta-cyclodextrins such as hydroxypropyl-beta-cyclodextrin, sulfobutylether-beta-cyclodextrin, maltoxyl-beta-cyclodextrin, and methylated cyclodextrins. Encompassed are alpha-cyclodextrins (6 glucopyranose units), beta-cyclodextrins (7 glucopyranose units), and gamma-cyclodextrins (8 glucopyranose units). Methylated cyclodextrins can improve aqueous solubility, dissolution rate, and bioavailability of cannabinoids.

The present disclosure provides a dermal patch (or buccal patch) comprising a dextrin where the dextrin is not complexed with a pharmaceutical agent, and a dermal patch (or buccal patch) comprising a dextrin where the dextrin is, in fact, complexed with a pharmaceutical agent.

In exclusionary embodiments, the present disclosure can exclude a formulation that comprises a cyclodextrin, or that comprises an alpha-cyclodextrin, or that comprises a beta-cyclodextrin, or that comprises a gamma-cyclodextrin. What can also be excluded is a device that comprises a cyclodextrin, such as an adhesive dermal patch comprising a dextrin or a buccal patch comprising a dextrin.

Matrices, Carriers, Binders, Tablets, Pills, Manufacturing Methods

A matrix, carrier, or binder, can include, e.g., hydrogel, polyethylene oxide, polyvinylpyrrolidone, hydroxypropyl cellulose, ethyl cellulose, methylcellulthose, alkylcelluloses, veegums clays, alginates, PVP, alginic acid, carboxymethylcellulose calcium, microcrystalline cellulose, polacrillin potassium, sodium alginate, corn starch, potato starch, pregelatinized starch, corn starch, modified starch, carnuba wax, montmorrilonite clays such as bentonite, gums, shellac, agar, locust bean gum, gum karaya, pecitin, tragacanth, and the like. In exclusionary embodiments, what can be excluded is one or more of the above polymers, clays, waxes, hydrogels, starches, and gums. A polyol can be used, for example, as a carrier. Polyols include propylene glycol and glycerol and the preferred (poly) alkoxy derivatives include polyalkoxy alcohols, in particular 2-(2-ethoxyethoxy) ethanol (Transcutol®).

Gums suitable for buccal tablets are disclosed in U.S. Pat. No. 4,829,056, which is incorporated by reference in its entirety. Lozenges and sublingual pills are provided, and these can comprise one or more of sodium phosphate, potassium phosphate, guar gum, gum arabic, locust bean gum, xanthan gum, carrageenan, carob gum, ghatti gum, pectin, tragacanth gum, acacia gum, mannitol, sorbitol, lactose, modified lactose, maltitol, mannitol, magnesium stearate, hydroxypropylmethylcellulose film, non-crystallizing sugar, or non-crystallizing sugar alcohol.

Matrix can be manufactured by melt-granulation, melt-extrusion, using particulates, granules, bilayers, plasticizers, and the like (see, US2016/0151502 of Wright). Patch can be made with silicone adhesives disposed on a substrate, copolymers, block polymers, tackifying resins, hot melt coating processes (see, US2014/0349108 of Fung). Patch can be made with backings, release liner, pressure sensitive adhesives, silicone gel adhesives (see, US2014/0287642 of Kumar). Dermal patch, buccal patch, tablets, can be made with excipient, disintegrant, swelling agent, films, binders, and the like (US2014/0079740 of Salama). Each of these patent documents is incorporated herein by reference in its entirety. Hot-melt extrusion, granules, tablets, transmucosal patches, transdermal patches, and methods of manufacture are detailed (Crowley et al (2007) Drug Development Industrial Pharmacy. 33:909-926; Repka et al (2007) Drug Development Industrial Pharmacy. 33:1043-1057).

Regarding sublingual tablets, sublingual pills, and sublingual strips, equipment for compressing granules, for applying coatings and lubricants, are available (see, US2010/0233257 of Herry). Regarding sublingual tablets and buccal tablets, formulas involving, e.g., cross-linked carboxymethylcellulose, lactose, microcrystalline cellulose, binding liquids, and equipment such as drier, mixer-granulator, compressor, are disclosed (see, e.g., U.S. Pat. No. 9,308,212). Penetration enhancers, fillers, binders, carriers, equipment for molding and solidifying sublingual tablets are disclosed (U.S. Pat. No. 9,220,747 of Gould). Each of these patent documents is incorporated herein by reference in its entirety.

Apertures and Pores

The present disclosure can encompass films, sheets, layers, membranes, and the like that have a plurality of apertures or pores. In some aspects, the apertures or pores have an average diameter of 20 nm, 40 nm, 50 nm, 100 nm, 200 nm, 300 nm, 400 nm, 500 nm, 600 nm, 800 nm, 0.001 mm, 0.002, 0.005 mm, 0.010 mm, 0.015 mm, 0.020 mm, 0.025 mm, 0.030 mm, 0.040 mm, 0.050 mm, 0.075 mm, 0.10 mm, 0.20 mm, 0.30 mm, 0.40 mm, 0.50 mm, and the like. Also, the pores can have a diameter range where the range is bracketed by any two of these values. In other aspects, the apertures or pores have a diameter in the range of 20-40 nm, 40-60 nm, 60-80 nm, 50-100 nm, 100-200 nm, 200-400 nm, 400-600 nm, 600-800 nm, 800-1,000 nm, 0.001-0.002 mm, 0.001-0.005 mm, 0.005-0.010 mm, 0.010-0.020 mm, 0.020-0.040 mm, 0.025-0.050 mm, 0.050-0.075 mm, 0.075-0.10 mm, 0.10-0.20 mm, 0.20 mm-0.40 mm, 0.25-0.50 mm, 0.50-0.75 mm, 0.50-1.00 mm, 0.1-0.2 mm, and so on. In exclusionary embodiments, the present disclosure can exclude films, sheets, layers, and the like, that have apertures or pores having any of the above average values, or that are describable by any of the above ranges.

Porous membranes can take the form of hydrophilic porous membranes and hydrophobic porous membranes, without implying any limitation. Hydrophobic membranes, such as hydrophobic polyethylene (PE) membranes, can be made more hydrophilic by alcohol or surfactants (see, WO2010/072233 of Calis). Pores in membranes of the present disclosure can have an average diameter of about 5 micrometers, about 10, about 15, about 20, about 25, about 30, about 40, about 50, about 60, about 70, about 80, about 90, about 100, about 110, about 120, about 130, about 140, about 150, about 160, about 170, about 180, about 190, or about 200 micrometers, and the like. Also, pores in the membranes can have an average diameter somewhere in the range 5-20 micrometers, 20-40 micrometers, 40-60 micrometers, 60-80 micrometers, 80-100 micrometers, 100-120 micrometers, 120-140 micrometers, 140-160 micrometers, 160-180 micrometers, 180-200 micrometers, and so on. In exclusionary embodiments, the present disclosure can exclude any membrane that is characterized by one of the above “about” values or that is characterizable by one of the above ranges.

For any given film, sheet, or layer, and the like, the area of a plurality of apertures or the area of a plurality of pores can occupy about 1%, about 2%, about 4%, about 6%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, and the like of the surface area. In exclusionary embodiments, the present disclosure can exclude any film, sheet, or layer, where the area does not occupy one or more of the given percentage values, or where the area does not occupy a range between any two of the above given percentage values. The above parameters also can apply to a film, sheet, or layer, with perforations, where the value of the area for the perforation is measured flush with a surface of the film, sheet, or layer.

Solubilizers and Surfactants

Solubilizers such as detergents, surfactants, organic solvents, and chaotropic agents, are available for the present disclosure. These can be one or more of, polyethylene glycol (PEG), propylene glycol, dibutyl subacetate, glycerol, diethyl phthalate (phthalate esters), triacetin, citrate esters-triethyl citrate, acetyltriethyl citrate, tributyl citrate, acetyltributyl citrate, benzyl benzoate, sorbitol, xylitol, bis(2-ethyllhexyl) adipate, mineral oil, polyhydric alcohols such as glycerin and sorbitol, glycerol esters such as glycerol, triacetate; fatty acid triglycerides, polyoxyethylene sorbitan, fatty acid esters such as TWEENS, polyoxyethylene monoalkyl ethers such as BRIJ series and MYRJ series, sucrose monoesters, lanolin esters, lanolin ethers. These are available from Sigma-Aldrich, St. Louis, Mo. In exclusionary embodiments, what can be excluded is any composition, formulation, dermal patch, and methods that comprise one or more of these solubilizers or surfactants.

The present disclosure can encompass compositions, formulations, devices, and methods, that comprise one or more surfactants, such as, sorbitan trioleate, sorbitan mono-oleate, sorbitan monolaurate, polyoxyethylene (20) sorbitan monolaurate, polyoxyethylene (20) sorbitan monooleate, oleyl polyoxytheylene (2) ether, stearyl polyoxyethylene (2) ether, lauryl polyoxyethylene (4) ether, block copolymers of oxyethylene and oxypropylene, diethylene glycol dioleate, tetrahydrofurfuryl oleate, ethyl oleate, isopropyl myristate, isopropyl palmitate, glyceryl monooleate, glyceryl monostearate, glyceryl monoricinoleate, cetyl alcohol, stearyl alcohol, cetyl pyridinium chloride, olive oil, glyceryl monolaurate, corn oil, cotton seed oil, and sunflower seed oil. In exclusionary embodiments, the present disclosure can exclude one or more of the above chemicals, and can also exclude a composition, formulations, device, and method that comprises any of the above chemicals.

Buffers and pH Values

The present disclosure can include formulations that contain a buffer with a pKa, as measured at room temperature, such as boric acid (pKa 9.2), CHES (pKa 9.5), bicine (pKa 8.3), HEPES (pKa 7.5), MES (pKa 6.1), MOPS (pKa 7.2), PIPES (pKa 6.8), Tris (pKa 8.1), imidazole (pKa 6.9), glycine (pKa 2.3), acetate (pKa 4.7), citrate (pKa 6.4), phosphate (pKa 7.21, 2.16, 12.32), malate (pKa 5.13), cacodylate (pKa 6.27), and the like. Also, the present disclosure can exclude formulations that include one or more of the above buffers, and can exclude a device that comprises one of these formulations. Without regard to any buffer, the present disclosure provides a formulation, or provides a component of a formulation, that has a pH value, as measurable at room temperature, of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, and the like. In exclusionary embodiments, the present disclosure can exclude a formulation, or can exclude a component of a formulation, that has a pH value, as measurable at room temperature, of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, and the like. The pH of component can be measured as pure component, that is, prior to combining with other components to generate formulation.

Buccal Patches, Sublingual Patches, and Related Pills, Tablets, and Strips

The present disclosure encompasses patch-based delivery systems for use in the mouth. In the mouth, regions for drug delivery include sublingual mucosa (area beneath the tongue) and the buccal mucosa (inner lining of the cheeks). Buccal administration of low water-solubility pharmaceuticals can be enhanced by formulating pharmaceutical in combination with a surfactant, or as a complex with hydrophilic cyclodestrins, or by using a nanosuspension (particle diameter in the nanomolar range, such as 50 nm to 150 nm) (see, Rao et al (2011) Int. J. Nanomedicine. 6:1245-1251). Nanoparticles can be made by milling, homogenization, or ultrasonication.

Buccal pouch is space between the cheek and the gums. Buccal dosage forms are inserted into the buccal pouch (see, U.S. Pat. No. 8,735,374 of Zerbe, which is incorporated herein in its entirety). Buccal patch can include an emulsifier that, when exposed to water, results in hydration-induced formation of an emulsifier. Emulsion can form spontaneously, that is, without much energy supply or without shearing forces, when water contacts the emulsifier. When placed against the gums, saliva drawn into the buccal patch can be the source of water. Self-emulsifying agent enhances the tendency of the formulation to adhere to the mucosal surface, thus promoting absorption of pharmaceuticals such as cannabinoids (see, U.S. Pat. No. 7,709,536 of Dam and U.S. Pat. No. 8,642,080 of Bender, each of which is incorporated herein by reference in its entirety).

This describes solvent casting and direct milling methods of manufacture. Without implying any limitation, buccal patch can consist of two laminates, with an aqueous solution of an adhesive polymer being cast on an impermeable backing sheet. One type of adhesive film can comprise an alcoholic solution of hydroxypropyl cellulose and organic acids. This adhesive film stays in place for at least 12 hours, even in the presence of fluids. Adhesive patches can be made by solvent casting or by direct milling. In solvent casting, all excipients and the drug are dispersed in an organic solvent and coated on a sheet of release liner. After solvent evaporates, a thin layer of protective material is laminated on the sheet of coated release liner to form a laminate. The laminate is then cut into patches (Koyi and Khan (2015) Buccal patches: A review. Int. J. Pharmaceutical Sciences Res. 4:83-89).

In direct milling, patches are created without using solvents. Drug and excipients are mixed by direct milling or by kneading, usually without any liquids present. After milling, the material is rolled on a release liner. A backing layer is then applied. Direct milling avoids the problem of residual solvents (Koyi and Khan (2015) Buccal patches: A review. Int. J. Pharmaceutical Sciences Res. 4:83-89).

The concerns solvent casting method and hot melt extrusion method. Without implying any limitation, buccal film can be made by solvent casting method and by hot melt extrusion method. Solvent casting involves dissolving water-soluble polymers to form viscous solution. Excipients are dissolved into solvent to give clear viscous solution. Then, both solutions are mixed (solution of water-soluble polymers; excipient solution) and then cast as a film, and then allowed to dry. This concerns hot melt extrusion. The drug or combination of drugs is in a dry state, and it is filled in a hopper, mixed, heated, and then extruded in a molten state. The molten mass that is formed is used to cast a film (Madhavi et al (2013) Buccal film drug delivery system—an innovative and emerging technology. J. Mol. Pharm. Org. Processing Res. Vol. 1, Issue 3 (6 pages)).

Without implying any limitation, mucoadhesive patches can be made by dissolving polymers in a solvent to produce a viscous solution. The polymers can be hydroxypropylmethyl cellulose (HPMC) E5LV and Carbopol® 940P. Polyethylene glycol 1000 can be included as a plasticizer. The solvent can be ethanol: chloroform (50:50). After creating of the viscous solution, drug can be dispersed in it. Then, the solution can be poured into molds for casting and dried for 24 hours. After drying, patches can be cut, for example, at 2 cm×2 cm. Each of the patches can contain, for example, 2 mg drug, 20 mg HPMC, 0.4 mg Carbopol, and 17 mg PEG100 (wt/vol) (see, Priya et al (2011) J. Pharm. Res. 3:56-65).

Laboratory Tests for Assessing Characteristics of Buccal Patches

Film thickness can be measured using puncture test and texture analyzer, such as Instron® 3366-2716015, Germany (see, Priya et al (2011) J. Pharm. Res. 3:56-65). Franz diffusion cell can measure drug release and permeation, with in vitro tests (Cavallari et al (2013) Eur. J. Pharm. Biopharm. 83:405-414; Technical Brief 2009, volume 10. Development and validation of in vitro release testing methods for semisolid formulations (Particle Sciences, Bethlehem, Pa.). Patch thickness can be measured with a screw gauge, where thickness can be measured at various different spots on the patch. To measure surface pH, patch can be allowed to swell for 2 hours on the surface of an agar plate (2% w/v), and the pH then measured with pH paper. Swelling can be measured by taking the weight each hour for six hours, after placing patch on an agar plate (see, Verma et al (2014) Effect of novel mucoadhesive buccal patches of carvediol on isopenaline-induced tachycardia. J. Adv. Pharm. Technol. Res. 5:96-103). Residence time measured time that patch adheres to a mucosal membrane, where patch is glued to a substrate, with repeated up-and-down movement of the substrate until the patch detaches (see, Ismail et al (2003) Design and characteristics of mucoadhesive buccal patches containing cetyl pyridinium chloride. Acta Pharm. 53:199-212.

Emulsions and Self-Emulsifying Agents

The present disclosure provides emulsions, emulsifying agents, self-emulsifying agents, creams, and lotions. The following provides examples of self-emulsifying agents. Self-emulsifying drug delivery systems (SEDDS) and self-nano-emulsifying drug delivery systems (SNEDDS) have been reviewed (see, Cherniakov et al (2015) Expert Opin. Drug Deliv. 12:1121-1133). Self-emulsifying agents include glycerol monostearate, glycerol monooleate, and Cremophor RH40®. Cremophor RH40@ is polyoxyl 40 hydrogenated castor oil. Cremophor EL® is polyoxyl 35 castor oil. These chemicals can be obtained from BASF Aktiengesellschaft, Ludwigshafen, Germany. In one aspect, the present disclosure can include formulations that comprise a self-emulsifying agent. In another aspect, the present disclosure can exclude formulations, and can exclude devices, that comprise a self-emulsifying agent.

Solubilizer SL-11 is a self-emulsifying agent that provides a nanoemulsion suitable for containing a hydrophobic drug (NOF America Corp., Irvine, Calif.). Emulsion with particle size under 50 nanometers can be made by these steps: (1) Dissolve drug in a suitable solvent, such as ethanol; (2) Add the drug solution prepared in (1) to Solubilizer SL-11, thoroughly mix to completely dissolve the contents; (3) The drug/SL-11 solution with solvent is made; (4) Evaporate the solvent at 50 degrees for about 1 hour to remove the solvent, or remove the solvent under a nitrogen stream; (5) Concentrated solution of SL-11 and the drug is made; (6) Soft capsules can be prepared by using the concentrated solution in (5) (NOF America Corp., Irvine, Calif.).

The following provides another non-limiting example. According to Shah et al (1994) Int. J. Pharmaceutics. 106:15-23, self-emulsifying agents can be made with polyglycolyzed glycerides (PGG) with varying fatty acid and polyethylene glycol (PEG) chain lengths, where these produce the self-emulsification of oil in water. The quality of the resulting emulsions depends on the oil and emulsifier pair selected and on the concentration of PGG as the emulsifier. One suitable oil is an oil with a medium-chain triglycerides (caprylic acid and capric acid; Neobee M5®). Another suitable oil is peanut oil. With formation of the emulsion, parameters that can be measured include droplet size distribution, droplet polarity, the release rate of the drug and the oil/water partition coefficient of the drug. PGG was found to be a workable emulsifiers for use in self-emulsifying drug delivery systems (SEDDS) (Shah et al (1994) Int. J. Pharmaceutics. 106:15-23).

Yet another non-limiting example of a self-emulsifying agent is provided by Chambin et al (2004) Int. J. Pharmaceutics. 278:79-89. This describes a self-emulsifying system using Gelucire® 44/14, an excipient from the lauroyl macrogolglycerides family. The laboratory method involves producing a fine oil-in-water emulsion when introduced into an aqueous phase under gentle agitation as SEDDS. The advantage is improved solubility and bioavailability of poorly water-soluble drugs. Gelucire® 44/14 was ground into a powder by cryogenic grinding to produce solid oral dosage forms and resulting in formulations made of Gelucire® 44/14 and ketoprofen (90/10). Cryogenic grinding produced Gelucire® 44/14 in a powder form, where this process did not change its physical properties, emulsification capacities and dissolution performances of the formulation tested.

Devani et al (2004) J. Pharmacy Pharmacology. 56:307-316, provide the following example, using the drugs danazol and mefenamic acid. In self-emulsifying drug delivery systems (SEDDS), drugs are dispersed in an oil-surfactant mix that emulsifies on contact with water. Self-emulsifying systems can be based on the Labrafil family of polyglycolysed oils, using Tween 80 and Tween 20 as surfactants. The more hydrophilic oil-surfactant mixes showed a greater ease of emulsification and a lower particle size. A linear relationship was observed between the hydrophile-lipophile balance (HLB) of the mix and the solubility of both danazol and mefenamic acid, with more hydrophilic mixes showing greater drug solubility values.

This provides another non-limiting example. Zupancic et al (2016) Eur. J. Pharm. Biopharm. 109:113-121 described emulsifying properties of SEDDS composed of long chain lipids (LC-SEDDS), medium chain lipids (MC-SEDDS), short chain lipids (SC-SEDDS) and no lipids (NL-SEDDS). The drug, enoxaparin was incorporated via hydrophobic ion pairing in the chosen SEDDS. The average droplet size of chosen LC-SEDDS, MC-SEDDS and NL-SEDDS ranged between 30 and 40 nm. MC-SEEDS containing 30% Captex 8000, 30% Capmul MCM, 30% Cremophor EL and 10% propylene glycol and NL-SEDDS containing 31.5% Labrafil 1944, 22.5% Capmul PG-8, 9% propylene glycol, 27% Cremophor EL and 10% DMSO exhibited 2-fold higher mucus diffusion than LC-SEDDS. Both MC-SEDDS and NL-SEDDS showed sustained in vitro enoxaparin release. Orally administrated MC-SEDDS and NL-SEDDS yielded an absolute enoxaparin bioavailability of 2.02% and 2.25%, respectively.

Further regarding emulsions, emulsifying agent can be characterized by Hydrophilic Lipophic Balance (HLB). HLB system is numbered 1 to 20. HLB values of 3 to 6 are lipophilic and these form water-in-oil emulsions (see, Vadlamudi, Hyndavi, and Tejeswari (2014) Current Drug Discovery Technologies. 11:169-180). HLB values of 8 to 18 are hydrophilic and these form oil-in-water emulsions (see, Grimberg, Nagel, and Aitken (1995) Environ. Sci. Technol. 29:1480-1487).

Permeation Enhancers

The present disclosure provides permeation enhancers, for example, for use with a dermal patch or for a buccal patch. Suitable permeation enhancers include, 23-lauryl ether, Aprotinin, Azone, Benzalkonium chloride, Cetylpyridinium chloride, Cetyltrimethylammonium bromide, Cyclodextrin, Dextran sulfate, Lauric acid, Lauric acid/propylene glycol, Lysophosphatidylcholine, Menthol, Methoxysalicylate, Methyl oleate, Oleic acid, Phosphatidylcholine, Polyoxyethylene, Polysorbate 80, Sodium EDTA, Sodium glycocholate, Sodium glycodeoxycholate, Sodium lauryl sulfate, Sodium salicylate, Sodium taurocholate, Sodium taurodeoxycholate, Sulfoxides, and Alkyl glycosides (see, Shojaei et al (June 2001) Systemic drug delivery via the buccal mucosal route. Pharmaceutical Technology. Pages 70-81). Other enhancers of the present disclosure are 1-octanol, 2-ethylhexanol, 1-nonanol, 1-decanol, and so on.

Azone is 1-dodecylazacycloheptane-2-one. The present disclosure provides azone, as well as azone analogues, for use as a skin permeation enhancer (see, Chen et al (2014) Asian J. Pharmaceutical Sciences. 9:51-64). For example, an azone analogue can have a hydrophobic chain of varied chain lengths.

Permation enhancers of the present disclosure can be a biphasic composition having a lipid phase and a water phase. Lipid phase can be prepared by mixing isopropyl palmitate and lecithin. Water phase can be mixture of water and a surfactant. Surfactant can be Pluronic®, Pemulen®, Noveon®, or Carbopol®. Pemulen polymeric emulsifiers are high molecular weight, copolymers of acrylic acid and C10-C30 alkyl acrylate crosslinked with allyl pentaerythritol (Lubrizol, Inc. product sheet). Carbopol homopolymers are acrylic acid crosslinked with allyl sucrose or allyl pentaerythritol. Carbopol copolymer are acrylic acid and C10-C30 alkyl acrylate crosslinked with allyl pentaerythritol (Lubrizol, Inc. product sheet). Noveon® Polycarbophil, USP is a high molecular weight acrylic acid polymer crosslinked with divinyl glycol (Lubrizol, Inc. product sheet). Pluronic® polymers are block copolymers based on ethylene oxide and propylene oxide. They can function as antifoaming agents, wetting agents, dispersants, thickeners, and emulsifiers (BASF, Inc. product sheet). The present disclosure can exclude any formulation, composition, device, method, and such, that comprise one or more the molecules found in Pluronic®, Pemulen®, Noveon®, and Carbopol®.

PLOGel is “Pluronic Lecithin Organogel” (Pharmedica Enterprise, Selangor, Malaysia). PLOGel takes the form of an aqueous phase (240 mL poloxamer 407, potassium sorbate, water) and organic phase (60 mL lecithin, isopropyl palmitate, sorbic acid). The present disclosure can exclude any formulation, composition, device, method, and such, that comprise one or more of PLOGel, poloxamer 407, potassium sorbate, isopropyl palmitate, sorbic acid, lecithin, and the like.

In exclusionary embodiments, the present disclosure can exclude any formulation, composition, device, method, and such, that encompasses one of the above polymers, polymer compounds, and crosslinked polymer compositions.

Bioadhesive Materials

Bioadhesive polymer of the present disclosure, when swollen, creates a flexible network through with drug can diffuse. Bioadhesive material serves a matrix for retaining pharmaceutical agents, until patch is applied to the skin or to a mucosal surface of the consumer. Bioadhesive materials include, hydroxypropyl cellulose, carbopol, poly(vinyl pyrrolidone), sodium carboxymethyl cellulose, hydroxyethyl cellulose, polycarbophil, pectin, chitosan, xanthan gum, locust bean gum, hydroxypropyl methylcellulose, poly(vinyl alcohol), poly(isoprene), poly(isobutylene) (see, Shojaei et al (June 2001) Systemic drug delivery via the buccal mucosal route. Pharmaceutical Technology. Pages 70-81).

Nutraceuticals and Pharmaceuticals

The present disclosure provides formulations, emulsions, and the like, as well as buccal patches and dermal patches, where the formulation, emulsion, buccal patch, and dermal patch, contains one or more of vitamin B1, vitamin D3, vitamin B12, or vitamin C, optionally in combination with one or more cannabinoids. Also, the formulation, emulsion, buccal patch, and dermal patch, can contain sildenafil.

### Exclusionary Embodiments

The present disclosure can exclude a composition, formulation, dermal patch, methods of use, methods of manufacture, that comprise one or more of the following: capsaicin, 2-arachidonylglycerol, curcumin, glycerylmonooleate, glycerylmonostearate, lecithin, acacia gum, xylitol, carboxymethylcellulose, a self-emulsifying agents, glycerol monostearate, glycerol monooleate, Cremophor RH40®, Cremophor EL®, hydroxypropyl cellulose, carbopol, poly(vinyl pyrrolidone), sodium carboxymethyl cellulose, hydroxyethyl cellulose, polycarbophil, pectin, chitosan, xanthan gum, locust bean gum, hydroxypropyl methylcellulose, poly(vinyl alcohol), poly(isoprene), poly(isobutylene). The present disclosure can also exclude one or more of, 23-lauryl ether, Aprotinin, Azone, Benzalkonium chloride, Cetylpyridinium chloride, Cetyltrimethylammonium bromide, Cyclodextrin, Dextran sulfate, Lauric acid, Lauric acid/propylene glycol, Lysophosphatidylcholine, Menthol, Methoxysalicylate, Methyl oleate, Oleic acid, Phosphatidylcholine, Polyoxyethylene, Polysorbate 80, Sodium EDTA, Sodium glycocholate, Sodium glycodeoxycholate, Sodium lauryl sulfate, Sodium salicylate, Sodium taurocholate, Sodium taurodeoxycholate, Sulfoxides, and Alkyl glycosides. What can also be excluded is a formulation, composition, device, or method, that comprises pre-gelatinized starch, gelatinized starch, gelatinized corn starch, glycogelatin, alpha-tocopherol, glycogelatin, hemp oil, THC, CBD, gum acacia, sorbitol, xylitol, soy lecithin, a complex of two different gels (one with net negative charge and the other with net positive charge), and a compositions that comprise a solvent with a cosolvent.

For delivery of cannabinoids, for example, a system of solvent/cosolvent can be ethanol (solvent)/propylene glycol (cosolvent). Solvents can be anhydrous alcohol, ethanol, propanol, or isopropanol. Cosolvent can be propylene glycol or PEG. Ratio of solvent/cosolvent (by weight) can be about 5/95, about 10/90, about 15/85, about 20/80, about 25/75, about 30/70, about 35/65, about 40/60, about 45/55, about 50/50, about 55/45, about 60/40, about 65/35, about 70/30, about 75/25, about 80/20, about 85/15, about 90/10, about 95/5, and the like. In exclusionary embodiments, the present disclosure can exclude solvent/cosolvent compositions where the ratio is, 5/95, about 10/90, about 15/85, about 20/80, about 25/75, about 30/70, about 35/65, about 40/60, about 45/55, about 50/50, about 55/45, about 60/40, about 65/35, about 70/30, about 75/25, about 80/20, about 85/15, about 90/10, about 95/5, and the like.

### Further Exclusionary Embodiments

What can be excluded is a formulation with an ethanol content, by weight, of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%. Also, what can be excluded is a formulation with an ethanol content, by weight, that encompasses about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%. Moreover, what can be excluded is a formulation with an ethanol content, by weight, that encompasses (range that equals or range that includes) a range that is 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or 95-100%. What can also be excluded is a device that encompasses one or more of the above formulations.

The present disclosure can provide a formulation that comprises ethanol and propylene glycol (or glycerol monostearate, or glycerol monooleate, or monoglyceride, or diglyceride, or triglyceride, or PEG, or phospholipid, or surfactant), and where the ratio (weight/weight basis) is about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In exclusionary embodiments, what can also be excluded is a formulation that comprises ethanol and propylene glycol (or glycerol monostearate, or glycerol monooleate, or monoglyceride, or diglyceride, or triglyceride, or PEG, or phospholipid, or surfactant), and where the ratio (weight/weight basis) is about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5.

Formulations with specific concentrations, on a weight basis, of propylene glycol or of any other compound can be excluded. What can be excluded are formulations containing about 0.1%, of about 0.2%, of about 0.4%, of about 0.6%, of about 0.8%, of about 1.0%, of about 2%, of about 4%, of about 6%, of about 8%, of about 10%, of about 15%, of about 20%, of about 25%, of about 30%, of about 35%, of about 40%, of about 45%, of about 50%, and the like, of propylene glycol, polyethylene glycol (PEG), polyalkylene glycol, ethanol, emulsion (e.g., oil droplets in water, water droplets in oil, liposome suspension), colloid, solvent, penetration enhancer, stabilizing agent, solubilizing agent (e.g., surfactant, detergent), gelling agent (either in dry state or in hydrated state), hydrogel (either in dry state or in hydrated state), adhesive, or any other compound, can be excluded.

Also, what can be excluded are formulations that encompass (range that equals or range that includes) the range of 0-0.1%, 0-5%, 0-10%, 0-20%, 0-30%, 0-40%, 0-50%, 5-10%, 5-15%, 5-20%, 5-40%, 5-50%, 10-20%, 10-30%, 10-40%, 10-50%, 10-60%, 10-70%, 20-30%, 20-40%, 20-50%, 20-60%, 20-70%, 20-80%, 30-40%, 30-50%, 30-60%, 30-70%, 30-80%, 40-50%, 40-60%, 40-70%, 40-80%, 40-90%, 50-60%, 50-70%, 50-80%, 50-90%, 60-70%, 60-80%, 60-90%, 60-100%, 70-80%, 70-85%, 70-90% 70-95%, 70-100%, 80-85%, 80-90%, 80-95%, 80-100%, 85-90%, 85-95%, 85-100%, and the like, of propylene glycol, polyethylene glycol (PEG), ethanol, emulsion (e.g., oil droplets in water, water droplets in oil, liposome suspension), colloid, solvent, penetration enhancer, stabilizing agent, solubilizing agent (e.g., surfactant, detergent), gelling agent (either in dry state or in hydrated state), hydrogel (either in dry state or in hydrated state), adhesive, or any other compound. In another aspect, the present disclosure can include (encompass, comprise) a formulation, composition, device, or method that comprises one or more of the above chemicals, at any of the recited “about” values, and at any of the recited ranges.

Without implying any limitation, the present disclosure can exclude a composition that comprises one or more of the following compounds, and can also exclude a device that comprises one or more of the following compounds. What can be excluded is a compound that is, buprenorphine, clonidine, estradiol, fentanyl, granisetron, methylphenidate, nitroglycerin, oxybutynin, scopolamine, selegiline, testosterone, a vaccine, influenza virus vaccine, a mammalian hormone, a synthetic analogue of a mammalian hormone, a chemically modified mammalian hormone, lidocaine, estrogen, salicyclic acid, a contraceptive, rivastigmine, rotogotine, tulobuterol, adrenergic agonist, cholinesterase inhibitor, dopamine receptor agonist, oxybutynin, bupropion, varenicline, nicotine, antidepressant, smoking cessation drug, cholinsterase inhibitor, methylphenidate, buprenorphine, opioid analgesic agent, sumatriptan, antiviral drug, anti-retrovirus drug, mammalian steroid, chemical analogue of mammalian steroid, drug for attention-deficit hyperactivity disorder, and so on.

In embodiments, the present disclosure can exclude a reservoir-type device where backing does not directly contact reservoir; or where reservoir does not directly contact a hydrophilic porous membrane; or where hydrophilic porous membrane does not directly contact a release liner; or where reservoir does not contain all of: (1) a liquid carrier, (2) a gelling agent, and (3) CBD. Also, what can be excluded is a reservoir-type device that does not comprise all of the above.

In embodiments, what can be excluded is an adhesive polymer, or a device comprising an adhesive polymer, where the adhesive polymer reacts with amines. Also what can be excluded, is an adhesive polymer, or a device comprising an adhesive polymer, where the adhesive polymer has any free hydroxyl groups, where the adhesive polymer has over 1 free hydroxyl groups per 100 atoms of the adhesive polymer, where the adhesive polymer has over 5 free hydroxyl groups per 100 atoms of the adhesive polymer, where the adhesive polymer has over 10 free hydroxyl groups per 100 atoms of the adhesive polymer, where the adhesive polymer has over 20 free hydroxyl groups per 100 atoms of the adhesive polymer, and so on. For this exclusionary embodiment, the skilled artisan understands that any polymer consists of a large number of atoms, for example, about five thousand atoms.

In embodiments, what can be excluded is a monolith-type device where a backing is not in direct contact with a matrix of skin adhesive; where matrix of skin adhesive is not in direct contact with a releasable liner; where matrix does not comprise CBD; or all of the above.

What can also be excluded is a preparation, or a device comprising a preparation, where the preparation has over 1% gelling agent, over 2%, over 3%, over 4%, over 5%, over 6%, over 7%, over 8%, over 9%, over 10%, over 12%, over 14%, or over 16%, of gelling agent. Also, what can be excluded is a preparation, or a device comprising a preparation, where the preparation has under 1% gelling agent, under 2%, under 3%, under 4%, under 5%, under 6%, under 7%, under 8%, under 9%, under 10%, under 12%, under 14%, or under 16%, of gelling agent.

What can also be excluded is a preparation, or a device comprising a preparation, where the preparation has over 1% penetration enhancer, over 2%, over 3%, over 4%, over 5%, over 6%, over 7%, over 8%, over 9%, over 10%, over 12%, over 14%, or over 16%, of penetration enhancer. Also, what can be excluded is a preparation, or a device comprising a preparation, where the preparation has under 1% penetration enhancer, under 2%, under 3%, under 4%, under 5%, under 6%, under 7%, under 8%, under 9%, under 10%, under 12%, under 14%, or under 16%, of penetration enhancer.

In other embodiments, what can be excluded is a preparation, a composition, a device comprising a preparation, a device comprising a composition, where said preparation or composition has a CBD (or THC, or combined weight of CBD and THC) content by weight of under 1%, under 2%, under 3%, under 4%, under 5%, under 6%, under 8%, under 10%, under 12%, under 14%, under 16%, under 18%, under 20%, under 25%, under 30%, under 35%, under 40%, under 45%, under 50%, under 55%, under 60%, under 65%, under 70%, under 75%, and so on. Also, what can be excluded is a preparation, a composition, a device comprising a preparation, a device comprising a composition, where said preparation or composition has a CBD (or THC, or combined weight of CBD and THC) content by weight that is greater than 5%, greater than 6%, greater than 7%, greater than 8%, greater than 10%, greater than 12%, greater than 14%, greater than 16%, greater than 18%, greater than 20%, greater than 25%, greater than 30%, greater than 35%, greater than 40%, greater than 45%, greater than 50%, greater than 55%, greater than 60%, greater than 65%, greater than 70%, and so on. In embodiments, what can be excluded is a preparation, a composition, a device comprising a preparation, or a device comprising a composition, where the percent by weight is defined by one or more of the above “under” or “greater than” parameters. “Composition” can refer to, for example, matrix of a skin adhesive, or to fluid in hydrophilic porous membrane, and so on. Alternatively, the present disclosure can comprise one or more of the above compositions, as set forth by “under” parameters or “greater than” parameters.

Moreover, in embodiments what can be excluded is any device that does not include an occlusive system polymer film, that does not include a polyethylene occlusive polymer film, that does not include a PET occlusive polymer film, that does not include an occlusive polymer film made of both polyethylene and PET. Also, what can be excluded is a device that has an overlay patch, and a device that does not comprise an overlay patch.

In embodiments, polar organic liquid can comprise, or can exclude, one or more of methanol, ethanol, propanol, isopropanol, butanol, pentanol, acetic acid, propionic acid, butyric acid, valeric acid, caproic acid, caprylic acid, capric acid, lauric acid, myristic acid palmitic acid, stearic acid, palmitoleic acid, oleic acid, linoleic acid, linolenic acid, linear alkanes of 5, 6, 7, 8, 9, 10, 11, 12, 13, 14, 15, 16, 17, 18, 19, 20, 21, 22, 23, 24, or more carbons, branched chain alkanes with a backbone of 5, 6, 7, 8, 9, 10, 11, 12, 13, 14, 15, 16, 17, 18, 19, 20, 21, 22, 23, 24, or more carbons, linear alkenes (olefins) of 5, 6, 7, 8, 9, 10, 11, 12, 13, 14, 15, 16, 17, 18, 19, 20, 21, 22, 23, 24, or more carbons, branched chain alkenes (olefins) with a backbone of 5, 6, 7, 8, 9, 10, 11, 12, 13, 14, 15, 16, 17, 18, 19, 20, 21, 22, 23, 24, or more carbons, and so on. Alternatively, the present disclosure can comprise one or more of the above polar organic liquids.

The present disclosure can exclude a composition, sublingual pills, device, transdermal monolithic and reservoir patches, buccal patches, method, that comprises an essential oil, a plant oil, a vegetable oil, or a fish oil. Also, the present disclosure can exclude a composition, device, method, that comprises one or more terpenes. What can be excluded is a composition, device, method, that comprises one or more of peppermint oil, orange oil, lemon oil, cannabis oil, hemp oil, and so on. Also, what can be excluded is any composition, device, or method, that comprises one or more of alpha-bisabolol, borneol, alpha-caryophyllene, beta-caryophyllene, elemene (alpha, beta, gamma, or delta), limonene, camphene, camphor, delta-3-carene, caryophyllene oxide, alpha-cedreen, citral, eucalyptol, beta-eudesmol, eudesm-7(11)-en-4-ol, farnesene, fenchol, alpha-guaiene, geraniol, guaiol, germacrene B, guaia-1(10)-11-diene, humulene, alpha-humulene, isobomeol, linalool, menthol, myrcene, alpha-myrcene, beta-myrcene, nerol, cis-ocimene, trans-ocimene, alpha-phellandrene, alpha-pinene, beta-pinene, pulegone, sabinene, alpha-terpinene, alpha-terpineol, terpinolene, terpineol, thymol, trans-2-pinanol, selina-3,7(1)-diene, or valencene.

Also, what can be excluded is a formulation, composition, device, lozenges, or sublingual pill that comprises one or more of sodium phosphate, potassium phosphate, guar gum, gum arabic, locust bean gum, xanthan gum, carrageenan, carob gum, ghatti gum, pectin, tragacanth gum, acacia gum, mannitol, sorbitol, lactose, modified lactose, maltitol, mannitol, magnesium stearate, hydroxypropylmethylcellulose film, non-crystallizing sugar, or non-crystallizing sugar alcohol.

What can be excluded is any formulation, composition, device, method, and such, that comprises menthol and isopropyl myristated in one of the following ratios (weight/weight): 200/10, 180/10, 160/10, 140/10, 120/10, 100/10, 90/10, 80/10, 70/10, 60/10, 50/10, 40/10, 30/10, 20/10, 15/10, 10/10, and so on, or one of the following ratios: 10/10, 10/15, 10/20, 10/30, 10/40, 10/50, 10/60, 10/70, 10/80, 10/90, 10/100, 10/120, 10/140, 10/160, 10/180, 10/200, and so on. Also, what can be excluded are compositions defined by a range of any of the above two ratio values. Also, what can be excluded is any formulation, composition, device, method, and such, that comprises menthol and isopropyl myristated in one of the following ratios (weight/weight): about 200/10, about 180/10, about 160/10, about 140/10, about 120/10, about 100/10, about 90/10, about 80/10, about 70/10, about 60/10, about 50/10, about 40/10, 30/10, 20/10, 15/10, 10/10, and so on, or one of the following ratios: about 10/10, about 10/15, about 10/20, about 10/30, about 10/40, about 10/50, about 10/60, about 10/70, about 10/80, about 10/90, about 10/100, about 10/120, about 10/140, about 10/160, about 10/180, about 10/200, and so on. Also, what can be excluded arc compositions defined by a range of any of the above two ratio values.

Inhaling Embodiments

Aerosols and dry powder formulations for inhaling are available. See, Mitchell, Nagel, Wiersema, and Doyle (2003) AAPS PharmSciTech. 4(4) Article 54 (9 pages); Asai et al (2016) Pharm. Res. 33:487-497; Kopsch et al (2017) Int. J. Pharm. 529:589-596; Fisher and Sznitman (2017) Inhalation. 11:21-25. Vaporizers are available, for example, from Storz and Bickel (Tuttlingen, Germany), Arizer Tech (Waterloo, Canada), Organicex (Las Vegas, Nev.), and Elemental Technologies (Seattle, Wash.).

### EXAMPLES

### Example One. Pill Formulations

Sublingual pill formulation was developed and tested for the active ingredients, cannabidiol THC and sildenafil. The formulation of the pill was: disintegrating agent (9 grams); microcrystalline cellulose (24 grams); saccharin sodium (0.75 grams); Mannitol (100 grams); magnesium stearate (1.5 grams). Active ingredients: 15 grams (CBD, Sildenafil). Total Pill Weight (150.5 grams).

Terpenes were used in composition of THC sublingual pills in concentration of THC 10 mg and 5 mg of terpene per pill. We also formulated terpenes in the reservoir patch in mix with THC and CBD

Pill formulation was developed to meet the acceptable performance criteria such as: Hardness, Friability and Disintegration; Hardness (greater than 1.5 kG/cm²); Friability (less than 2%); Disintegration (less than 100 sec). Sublingual pills were made using a commercial press LFA Tablet Press DTP 25 (Dallas, Tex.).

The laboratory results for different sublingual tablets formulations were as follows (Table 1). Average Pill Weight (250 mg). Diameter 10.1 mm and Thickness 4.3 mm. Cannabidiol was sourced from hemp extract in crystalline form having purity 99.8% (0.00% THC).

TABLE 1
Sublingual Tablet Weight Average 250 mg.

| | | | Disinte- |
|---|---|---|---|
| | Friability | Hardness | gration |
| | % | Kg/cm² | Sec. |
| CBD 40 mg. | 0.2 | 3 | 36 |
| CBD 20 mg | 0.1 | 4 | 59 |
| CBD 20 mg., Melatonin 3 mg. | 0.3 | 3 | 45 |
| CBD 12.5 mg., THC 5 mg. | 0.2 | 3.5 | 120 |
| THC 8 mg. | 0.2 | 3.5 | 60 |
| THC 8 mg., Sativa Terpene 5 mg. | 0.5 | 2.0 | 70 |
| THC 8 mg., Indica Terpene 5 mg. | 0.5 | 2.0 | 70 |
| SILDENAFIL 20 mg. | 0.5 | 3.5 | 55 |

Suppliers: Disintegrating agent (Pharmaburst 500 from SPI Pharma); microcrystalline cellulose (Avicel 102 from FMC BioPolymer); saccharin sodium (Spectrum Chemical MFG. Corp.); mannitol (from RPI Research Products International); magnesium Stearate (Spectrum Chemical MFG. Corp.). Sildenafil and cannabinoids are available from, for example, Sigma-Aldrich, St. Louis, Mo. For testing pills and tablets, friability, hardness, dissolution, and disintegration can be assessed by equipment from Copley Scientific, Ltd., Nottingham, UK. Equipment includes Friability Tester Series FR (FR1000, FR2000, Friabimat SA400), disintegration tester (DTG1000, DTG2000, DTG4000), and dissolution apparatus (basket, paddle, paddle over disk, cylinder, and vertical diffusion cell (Franz cell)). Friability is the tendency for a tablet to chip, crumble, or break under compression.

### Example Two

PIB adhesive with tackifiers that improve adhesion to skin using acrylic pressure sensitive adhesive mixed in at 1-50%. Also use of cycloaliphatic hydrocarbon resins such as Escorez 5300® resins from Exxon Mobil. Adhesion to skin increased about 2 fold.

### Example Three

PIB adhesive with enhancers: at 3% of azone or oleic acid double the transdermal delivery of CBD from PIB.

### Example Four

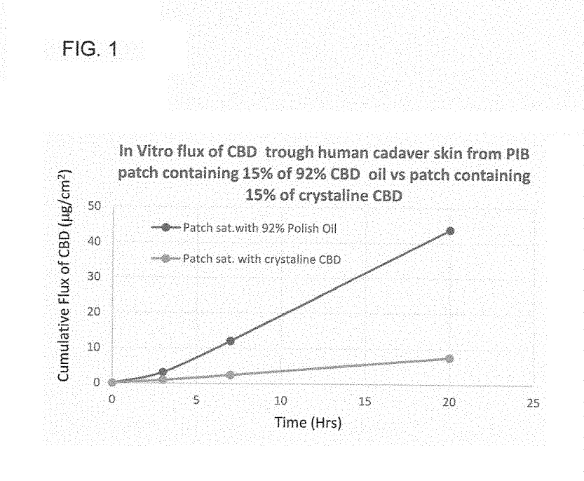

Use of hemp oil with CBD of high concentration 92% containing different terpenes improves transdermal delivery of CBD. This dermal patch delivers about 5 times greater flux of CBD than from adhesive matrix with crystalline CBD. FIG. 1 provides a graph of transdermal flux from matrix with crystalline CBD vs. matrix with hemp oil of 92% CBD (see, FIG. 1). In embodiments, this delivery is from a reservoir patch or from a monolithic patch.

### Example Five

Delivery of CBD and THC from semisolid hydrogels saturated with CBD and THC oils of high concentration of CBD and THC 80-95%. Oils are saturated in mix with EtOH/water in ratio 80/20 also with enhancers azone, oleic acid and limonene.

### Example Six

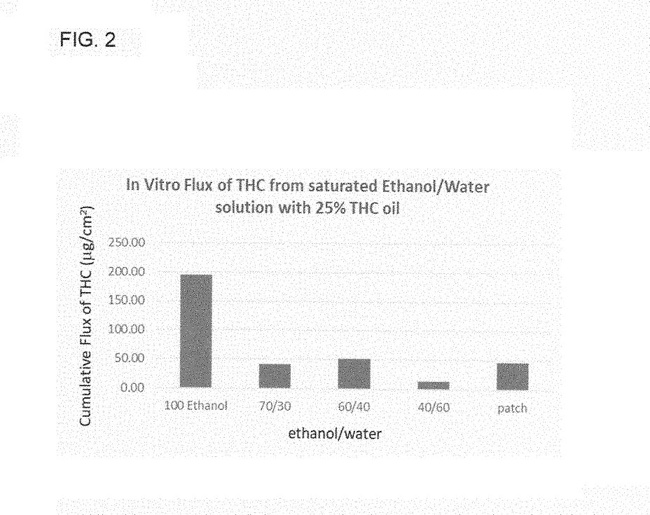

THC oil mixed with ethanol and with EtOH/water of different ratios in reservoir patch show the highest flux from pure ethanol. THC flux from ethanol is 4-times greater than from a monolithic patch. FIG. 2 provides a graph of transdermal flux (FIG. 2).

### Example Seven

CBD Patch with Menthol, Camphor and Salicylic acid.

### Example Eight

CBD patch with 0.01% Capsicum.

### Example Nine

CBD with nutraceutically active ingredients.

### Example Ten

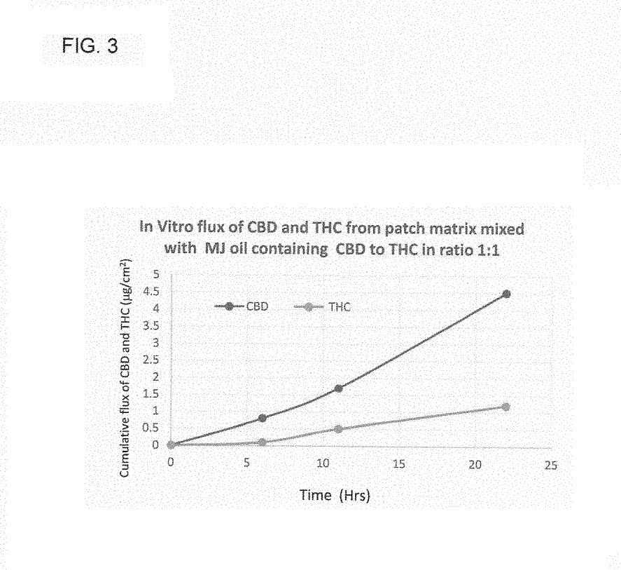

FIG. 3 discloses data from CBD/THC ratio 1/1 in patch produces 2/1 transdermal dose ratio. The disclosure provides a graph of transdermal flux (FIG. 3). The present disclosure provides a dermal patch, and a method for using the dermal patch, wherein the dermal patch is capable of delivering the following flux, as measurable with human cadaver skin and a Franz diffusion cell. As shown in FIG. 3, the dermal patch is capable of delivering a flux of CBD of at least about 4 micrograms per cm² over the course of about 20 hours and, simultaneously, a flux of at least about 1.0 micrograms per cm² THC over the course of about 20 hours, wherein the dermal patch comprises a patch matrix with an oil that contains CBD/THC in a 1:1 ratio (vol./vol.). Alternatively, the oil contains CBD/THC in a 1:1 ratio (wt./wt.).

### Example Eleven

The disclosure provides Melatonin Patch, Lidocaine Patch, Menthol, Camphor, Salicylic Acid Patch. Hang Over patch with Dihydromyricetin, Vitamin B1 patch, Vitamin D3 patch, Vitamin B12 patch, Vitamin C patch, Sildenafil sublingual pill, Sildenafil fast dissolving strip. Sildenafil buccal patch, Cannabidiol (CBD) sublingual pill, Cannabidiol fast dissolving strip, Cannabidiol buccal patch, and the like.

The present invention is not to be limited by compositions, reagents, methods, diagnostics, laboratory data, and the like, of the present disclosure. Also, the present invention is not be limited by any preferred embodiments that are disclosed herein.

Regarding FIG. 1, monolithic patch consisting of the adhesive layer and occlusive backing polyethylene film, Adhesive was PIB polymer with 15% of CBD oil containing 92% CBD and another patch has also PIB adhesive mixed with 15% of crystalline CBD. The following concerns reservoir patches. The composition inside the reservoir can have the consistency of a paste, but in an alternative embodiment, it can have the consistency of an oil, or it can have the consistency of a liquid, or it can have the consistency of a slurry. Parameters for FIG. 2 are as follows. Flux was allowed to occur for 24 hours, and FIG. 2 provides the value at 24 hours, For example, please describe the layers that were used to make up this patch. What was each layer made of? What was the volume of the reservoir? The layers are 30 cm square and the hydrogel is one cm cubed or one milliliter. The layers have microporous membrane with 5 millimeter pore size. The layers have occlusive heat seal around edges. There is a protective release liner. The embodiment can be made with or without silicone adhesive. FIG. 3 is monolithic patch with PIB adhesive and occlusive polyethylene backing, Adhesive was mixed in with 25% of marijuana extract (oil) in containing 28% of CBD and 32% of THC.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A composition capable of use in a buccal patch, a dermal patch, or a sublingual patch, tablet, capsule, pill, or strip, wherein the composition comprises one or more of:
(a) An acrylic adhesive with non-functionality and an adhesive with only OH-functionality, further comprising one of more of enhancers selected from azone, oleic acid, and dimethylsulfoxide (DMSO);
(b) A polyisobutylene (PIB adhesive) with tackifiers that improve adhesion to skin using acrylic pressure sensitive adhesive mixed in at 1-50%, optionally with a cycloaliphatic hydrocarbon resin;
(c) PIB adhesive with enhancers: at 3% of azone or oleic acid double the transdermal delivery from PIB. The disclosure provides a graph showing transdermal flux;
(d) Hemp oil with CBD of concentration 80-95% containing at least one terpene;
(e) A semisolid hydrogel that is saturated with cannabidiol (CBD) and tetrahydroxannabinol (THC);
(f) A semisolid hydrogel comprising an oil that consists essentially of CBD and THC (80-95%, wt/vol), in combination with ethanol/water (80/20, vol/vol), optionally with one or more enhancers selected from azone, oleic acid, and limonene;
(g) A semisolid hydrogel saturated with CBD and THC oils (80-95%, wt/vol), wherein the oil is mixed with EtOH/water (80/20, vol/vol), optionally with one or more enhancers selected from azone, oleic acid, and limonene; or
(h) A THC oil of THC (80-95%) mixed with 1-20% EtOH/water or with 1-10% EtOH/water (80/20, vol/vol) wherein including greater than 10% of ethanol is capable of lowering flux of THC delivery as determinable with a reservoir patch.

2. A buccal patch comprising a composition of claim 1.

3. A dermal patch comprising a composition of claim 1.

4. A sublingual sublingual patch, tablet, capsule, pill, or strip, comprising a composition of claim 1.

5. A method for applying the buccal patch of claim 2 to the buccal mucosa of a human subject, and allowing a cannabinoid to transit from the patch into an oral mucosa of the human subject.

6. A method for applying the dermal patch of claim 3 to skin of a human subject, and allowing a cannabinoid to transit from the buccal patch into the skin of the human subject.

7. A method for applying the sublingual patch, tablet, capsule, pill, or strip of claim 4, to the tongue of a human subject, and allowing a cannabinoid to transit from the sublingual patch, tablet, capsule, pill, or strip, into an oral mucosa of a the human subject.

8. A method for manufacturing the patch of claim 1, comprising the steps of combining THC, a film, an adhesive, and a backing, to generate an uncut patch, further comprising the uncut patch to produce a cut patch that is capable of applying to human skin or of applying to human buccal pouch.

9. A method for delivering cannabidiol (CBD) to human skin, wherein the method comprises the step of contacting a dermal patch with the human skin, and wherein the contacting is for at least one hour, and wherein the delivering results in passage of CBD through the skin,
wherein the method uses a dermal patch that comprises polyisobutylene (PIB) and a cannabis oil that contains about 15% cannabidiol (CBD) oil, and wherein the delivering is measurable by a system that comprises human cadaver skin and a Franz diffusion cell, wherein said method is capable of delivering one or both of:
(i) A cumulative flux of CBD of at least about 20 micrograms CBD per cm² of skin over a period often hours, and
(ii) A cumulative flux of CBD of at least about 40 micrograms CBD per cm² of skin over a period of twenty hours.

10. A method for delivering tetrahydrocannabinol (THC) to human skin, wherein the method comprises the step of contacting a dermal reservoir patch with the human skin, and wherein the contacting is for at least one hour, and wherein the delivering results in passage of THC through the skin,
the method resulting in cumulative flux of THC from a saturated ethanol/water solution with 25% THC oil, and wherein one or both of:
(i) The ratio of ethanol/water is about 70/30 and the flux is at least about 40 micrograms THC per cm², and
(ii) The ratio of ethanol/water is about 60/40 and flux is at least about 50 micrograms TI-C per cm².

11. A sublingual tablet of about 250 mg, wherein the sublingual tablet comprises CBD (20 mg) and melatonin (3 mg), and wherein the sublingual tablet has a friability of about 0.3%, a hardness of about 3 kg/cm2, and a disintegration of about 45 seconds.
